(19) United States
(12) Patent Application Publication
(10) Pub. No.: US 2023/0240888 A1
(43) Pub. Date: Aug. 3, 2023

(54) CURVED SHAPE MEMORY IMPLEMENT WITH CUTTING EDGE

(71) Applicant: Alcon Inc., Fribourg (CH)

(72) Inventor: Paul R. Hallen, Colleyville, TX (US)

(21) Appl. No.: 18/151,706

(22) Filed: Jan. 9, 2023

Related U.S. Application Data
(60) Provisional application No. 63/267,484, filed on Feb. 3, 2022.

(51) Int. Cl. A61F 9/007

(52) U.S. Cl. CPC A61F 9/007; A61B 2017/00867

(57) ABSTRACT

An eye surgery tool with an implement having a curved shape memory region that is straightenable for accessing an interior of an eye. Upon accessing the eye interior, the implement may take on a natural curve shape. The implement may include a cutting region or edge that is of a rigidity greater than that of the shape memory material constituting the majority of the shape memory region. In spite of the added rigidity, the shape memory material may serve as a master in a master-slave arrangement to curve the cutting region or edge as appropriate.

### BACKGROUND

Over the years, many dramatic advancements in the field of eye surgery have taken place. One of the more common eye surgery procedures is a vitrectomy. Vitrectomy is the removal of some or all of the vitreous humor from a patient's eye. In some cases, where the surgery is limited to removal of clouded vitreous humor, the vitrectomy may constitute the majority of the procedure. However, a vitrectomy may accompany cataract surgery, surgery to repair a retina, to address a macular pucker or a host of other issues.

Often a vitrectomy is accompanied by a variety of other procedures to address more specific eye features. That is, in addition to the described vitrectomy, other types of probes or implements may be utilized to address specific eye issues. The scenario may involve a degree of vitrectomy followed by the use of scissors, a scraper or other implement to directly interact with an eye feature. For example, cutting a film or particulate near the retina at the back of the eye may be desired. In this example, scissors may take the place of a vitrectomy probe for the sake of cutting followed by the use of a vitrectomy probe to achieve completed removal of the material cut by the scissors.

Scissors, forceps, scrapers and other implements that are tailored to such direct interaction with eye features as noted are generally quite straight, narrow and low profile devices. That is, even apart from conventional function, having an architecture that is fairly straight, narrow and small in profile may be beneficial for gaining access to the eye interior for the intervention. That is, reaching into the eye interior for the procedure is generally guided and supported by a preplaced cannula which has been positioned at the location of an incision through the pars plana. Thus, the called for implement may be securely advanced through to the interior of the eye to perform the surgical procedure. Of course, being guided through a straight channel of the cannula as described means that the implement would generally be of a similarly straight configuration.

Furthermore, in addition to being fairly straight, the low profile or narrow nature of the implement is also important in reaching the eye interior. Specifically, over the years, minimally invasive surgeries have employed smaller and smaller implements for increasingly precise surgical maneuvers. For example, vitrectomy probe needles, scissors or forceps that traditionally may have been about 23 gauge may be about 25 or 27 gauge with corresponding minimally sized cannulas for guidance. This translates to reducing the implement diameter from just under about 0.5 mm (millimeters) to less than about 0.4 mm.

Advancements in surgical technology have led to smaller and smaller incisions and instruments being a practical undertaking. Further advancements in instrument shape may also be helpful. For example, many eye surgical implements are straight and narrow, but the eye interior surface may be contoured. Taking the example of scissors, it may be that the surgeon would ideally seek to make a cut at the back of the eye from an angle that more closely matches the concave structure of the back of the eye. The surgeon may advance the implement to the back of the eye near the retina and be faced with a nearly perpendicularly oriented pair of scissors with respect to a retinal feature to be cut. The surgeon has some degree of play in terms of orienting the scissors about a pivot point of the preplaced cannula that has guided the scissors to the eye interior. However, the degree of pivot may be small and unlikely to allow the surgeon to easily make any cut that is more parallel to the retinal surface at the back of the eye.

The option of utilizing much larger cannulas or other form of surgical access to allow for the use of curved or bent scissors may be possible, but smaller inscisions are generally desired. Therefore, surgeons may instead be left with using straight scissors and other implements to address features at the back of the eye from a challenging, nearly perpendicular interface with the features.

### SUMMARY

A curved instrument for eye surgery is disclosed. The instrument may include a handle for surgical manipulation outside of a patient's eye. An implement coupled to the handle may be provided for reaching an interior of the eye through a preplaced cannula at an outer surface of the eye. A curved memory shaped region of the implement corresponding to a curved shape surface defining an interior of the eye is provided. Once more, this region is straightenable for traversing the cannula and returnable to the curved shape upon reaching the eye interior. The shaped memory region also includes a durable cutting edge for contacting a feature at the curved shape surface.

### BRIEF DESCRIPTION OF THE DRAWINGS

FIG. 1 is a perspective view of a tool for eye surgery employing an embodiment of a curved shape memory implement.

FIG. 2A is an enlarged view of cross-section of a curved shape memory region of the implement of FIG. 1 taken from 2-2 of FIG. 3A.

FIG. 2B is an enlarged view of an alternate embodiment of curved shape memory region for an implement similar to that of FIG. 1.

FIG. 3A is an enlarged perspective and cross-sectional view of the curved shape memory implement of FIG. 1 traversing a cannula placed at an eye.

FIG. 3B is an enlarged perspective and cross-sectional view of the implement of FIG. 3A passing the cannula and expanding to the curved memory shape of FIG. 1.

FIG. 4 is an enlarged and cross-sectional view of the implement of FIG. 3B reaching toward a curved surface defining an eye interior for a surgical procedure thereat.

FIG. 5 is a flow-chart summarizing an embodiment of utilizing a surgical tool with a curved memory shape implement to perform surgery in an eye.

### DETAILED DESCRIPTION

In the following description, numerous details are set forth to provide an understanding of the present disclosure. However, it will be understood by those skilled in the art that the embodiments described may be practiced without these particular details. Further, numerous variations or modifications may be employed which remain contemplated by the embodiments as specifically described.

Embodiments are described with reference to certain types of vitreoretinal procedures carried out with a unique implement. In particular, a procedure in which scissors are utilized to address issues at the back of a patient's eye is illustrated. Of course, a variety of other maneuvers may be carried out with the implement and others over the course of a single intervention. Regardless, so long as the implement is outfitted with a unique shaped memory region that allows for a linear profile to give way to a curved shape upon gaining access to the eye interior, appreciable benefit may be realized.

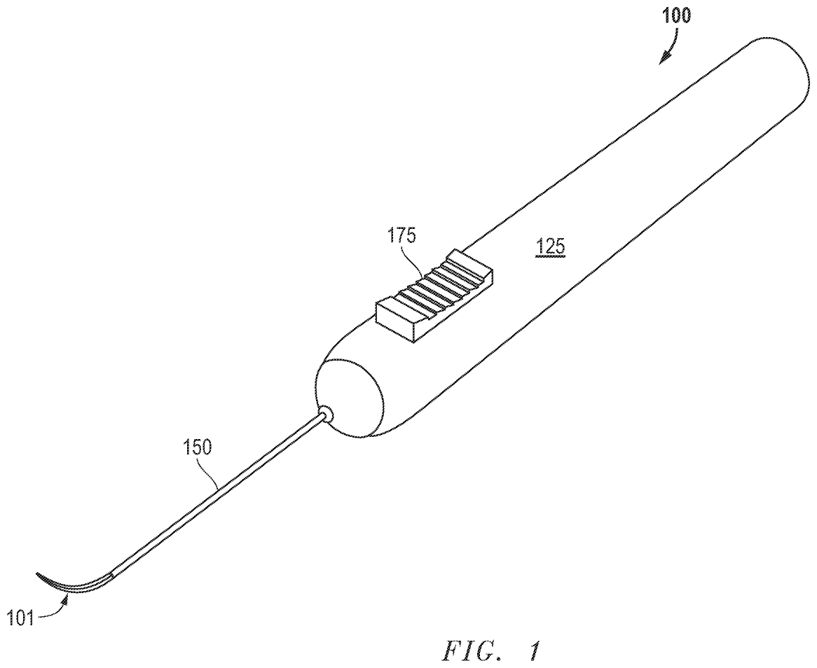

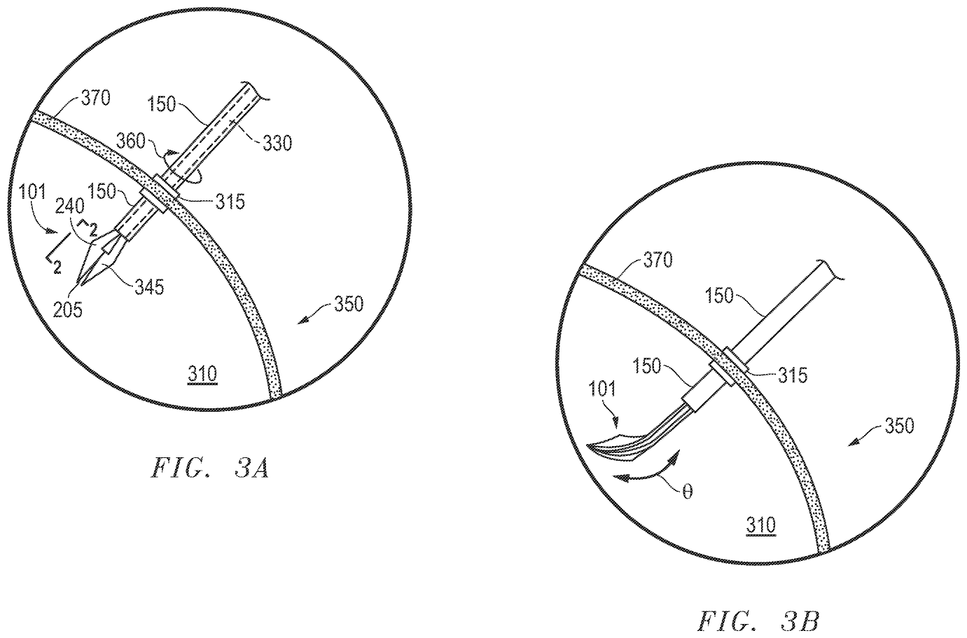

Referring now to FIG. 1, a perspective view of a tool 100 for eye surgery is illustrated that employs an embodiment of a curved shape memory implement 101. With added reference to FIGS. 3A and 3B, the tool 100 includes a handle 125 for a surgeon to hold along with a sleeve 150 to facilitate the introduction of the implement 101 into an eye 350. The handle 125 may also serve as a housing for various components such as those playing a role in directing the implement 101. For the embodiments illustrated herein, the sleeve 150 is utilized as an aid to the noted introduction into the eye 350. However, it should be noted that the sleeve 150 is not necessarily required as is detailed further herein. Further, in the embodiment shown, the tool 100 includes an actuator 175 at the handle 125 for actuation of the implement 101. For example, in one embodiment, the implement 101 includes scissors 240, 345 for opening and closing as directed by the depicted actuator 175. The actuator 175 may be a slider, button, lever, dial or any suitable actuating means for the application. This may include electro/mechanical actuating means as well.

As indicated, the implement 100 is referred to as a shaped memory implement. For the embodiments herein, this means that the implement 100 includes a region that may be naturally curved, for example, along an arc with an angle (θ) as more specifically illustrated in FIG. 3B. Reference to the angle (θ) and arc shape are not meant to infer any particular degree of angle. Rather, the angle (θ) would be dependent on the size of the curved shape memory region and the arc shape thereof may be consistent with what would constitute a completed circle were the shape to be continuous. Regardless, this region may also be conformable to an elongated or straightened character such as for positioning in the sleeve 150 or within a cannula 315 (again see FIGS. 3A and 3B).

The terms “shape memory” or “shaped memory” as used herein are not meant to infer that the implement is of a material that must reach a certain temperature or environmental condition in order to straighten or return to curved form. Rather, the terms are meant to highlight that the implement 101 includes a region of a superelastic material such as a nickel titanium alloy (e.g., Nitinol) or other suitable material which exhibits superelastic and shape memory properties. Thus, a significant amount of deformation may take place while still allowing the curved region of the implement 101 to return to the illustrated curved form as detailed below.

Continuing with reference to FIG. 1, a shape memory curved region of the implement 101 means that while naturally curved as illustrated, the curve in the implement 101 may be straightened and return to curved form as the application warrants. For example, the sleeve 150 may be of a stainless or other more rigid material by contrast. Thus, prior to emerging from the sleeve 150, the implement 101 may be of a straightened configuration within the sleeve 150 that gives way to the shape memory curve illustrated upon extending from within the sleeve 150.

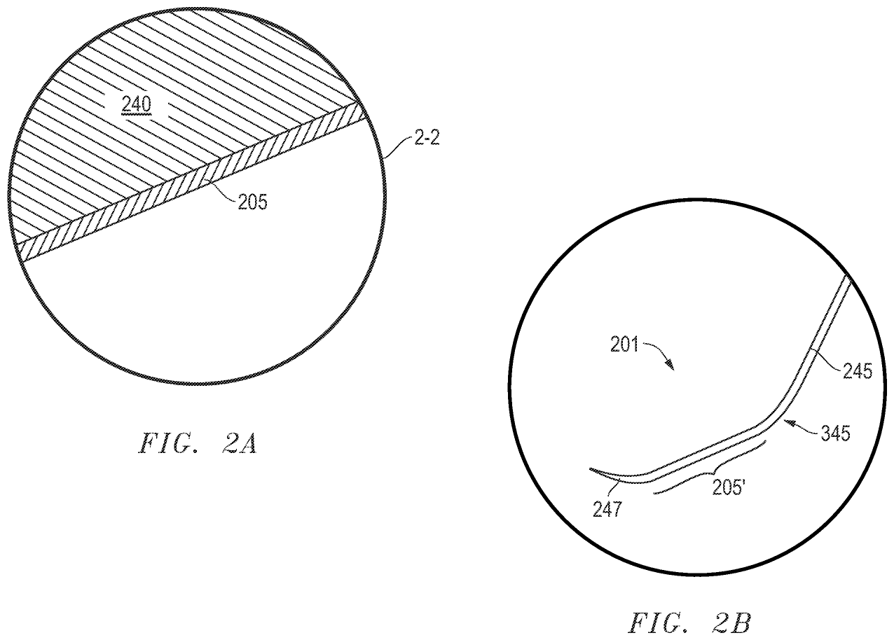

Referring now to FIG. 2A, an enlarged view of a cross-section of a curved shape memory region of the implement 101 of FIG. 1 is illustrated that is taken from 2-2 of FIG. 3A. Continuing with the added reference to FIGS. 1 and 3A, the region of the implement 101 includes blades 240, 345 of a pair of scissors. Specifically, FIG. 2A depicts a cross-section of one of the blades 240 that is made up of a shape memory material such as the above noted Nitinol. However, the blade 240 also includes a rigid edge portion 205 that is non-shape memory in character. For example, the edge portion 205 may be of a stainless steel or other suitable cutting edge material(s). Indeed, the edge of the adjacent blade 345 of FIG. 3A might also be of the same or similar rigid material.

FIG. 2A is an enlarged view and in cross section. Thus, no shape memory arc angle (θ) is apparent as illustrated for the implement 101 in FIGS. 1 and 3B. For added context, FIGS. 1 and 3B reveal the shape memory curve of the implement 101, whereas FIG. 3A depicts the implement 101 as it emerges from the above described sleeve 150. Thus, in FIG. 3A, the implement 101 has not yet been fully freed from the constraints of the sleeve 150 and has not yet taken on the illustrated curved shape of FIGS. 1 and 3B.

Continuing with reference to FIG. 2A, it is apparent that a substantial majority of the illustrated blade 240 constitutes a shape memory material in contrast to its more rigid edge 205. In keeping with the example of a shape memory material in the form of Nitinol and an edge 205 of stainless steel, the comparative rigidity of the edge 205 would still be insufficient resistance to the implement blade 240 taking on the arced curve (e.g., as illustrated in FIGS. 1 and 3B). Stated another way, once the shape memory region of the implement 101 is freed from the confines of the sleeve 150 (and/or the cannula 315), it is of sufficient shape memory force so as to overcome any rigid edge 205 resistance to attaining the arc (θ) as illustrated in FIGS. 1 and 3B.

Joining the materials of the shaped memory blade 240 with its more rigid edge 205 may involve standard welding techniques that are utilized with shape memory alloys such as Nitinol. Preferred welding techniques may include autogenous welding, microplasma arc welding, tungsten inert gas (TIG) welding, laser beam welding, electron beam welding, pressure welding and capacitor discharge welding.

Of course, these joining or welding techniques may be applied to the adjacent materials 205, 240, generally following application of a shape memory creation technique applied to the main body of the illustrated blade 240. More specifically, before adding the illustrated edge 205, the shape memory material may be cast by way of vacuum arc or induction melting of different material constituents (e.g. nickel and titanium). This melting generally occurs at 400° C.-500° C. and below any crystallizing level of heat for a period. Shaping with the materials still hot followed by rapid cooling via water or air may take place in order to attain the desired shape such as the arc (θ) illustrated in FIGS. 1 and 3B.

Referring now specifically to FIG. 2B, an enlarged view of an alternate embodiment of a curved shape memory region for an implement 101 similar to that of FIG. 1 is illustrated. However, in this embodiment, the curved region 201 of the implement is discontinuous. That is, as opposed to defining a particular consistent arc shape as shown in FIGS. 1 and 3B, a flat region 205′ is supplied to the depicted blade 345. Thus, in this instance, the cutting edge for the scissors implement 101 is not supplied by adding an elongated edge 205 to a single block of shape memory material 240 as illustrated in FIG. 2A. Instead, a conventional flat edge of stainless steel or other suitably rigid material is used to make up the flat region 205′ for cutting character. This region 205′ is then joined or welded as described above to separately created shape memory material segments (e.g. 245, 247). Of course, in this embodiment, the illustrated flat region 205′ would pair with a similar flat region of an adjacent scissors blade. In one embodiment, the depicted flat region 205′ as well as that of an adjacent scissors blade would span less than about 5 mm and be well suited for use within an eye interior 310 as illustrated in FIGS. 3A and 3B.

Referring now to FIG. 3A is an enlarged perspective and cross-sectional view of the curved shape memory implement 101 of FIG. 1 is shown traversing a cannula 315 placed at an eye 350 for micro-invasive vitreoretinal surgery (MIVS). As detailed further below, the implement 101 is guided through the eye 350 at an offset sclera 370 location to reach the eye interior 310. In the embodiment shown, a sleeve 150 is used to breach the cannula 315. However, in other embodiments, the sleeve 150 may stop at the cannula 315 and function as a stiffening sleeve for support solely from the exterior of the eye 350. Alternatively, in another embodiment, there may be no sleeve 150 component at all. For this embodiment, a rod or mandrel 330 used to advance the implement scissors 101 as illustrated may be the sole supportive feature.

Referring now to FIG. 3B, an enlarged perspective and cross-sectional view of the implement 101 of FIG. 3A is shown passing the cannula 315 and expanding to the curved memory shape of FIG. 1 (e.g. taking on angled arc (θ) shape). In this view, the entire tool is shown rotated clockwise from the position illustrated in FIG. 3A (see arrow 360). That is, in FIG. 3A, the front or top view of the scissors implement is shown for discussion. However, for FIG. 3B, the rotation of arrow 360 takes place to present more of a side view and arc angle appearance (θ) of the now more fully extended implement 101. That is, for FIG. 3B, the implement 101 is now more fully advanced beyond the sleeve 150 and cannula 315 to allow for taking on the full arc shape within the eye interior 310.

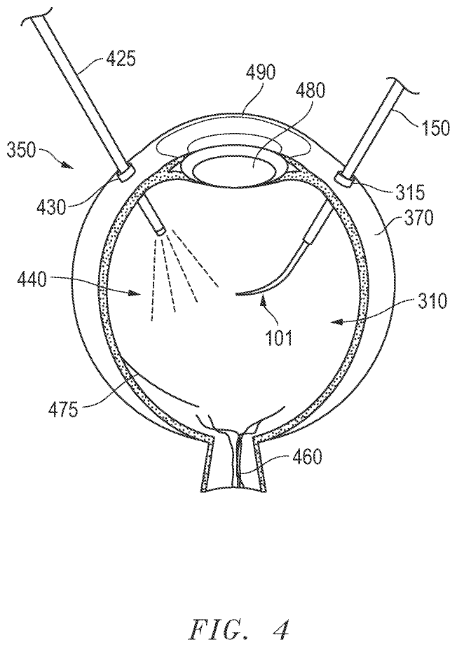

Referring now to FIG. 4, an enlarged and cross-sectional view of the implement 101 of FIG. 3B is shown reaching toward a curved surface defining an eye interior 310 for a surgical procedure thereat. Indeed, the entire interior 310 of the eye 350 is defined by a curved surface such as choroid at the middle of the eye interior 310 or the retinal surface closer to the back. Indeed, the latter includes an ophthalmic tissue membrane 475 to be cut by the implement scissors 101.

Noting that the diameter of the eye 350 for a human is likely between about 24 mm and 34 mm as defined by the noted inner surface, a corresponding curved profile of the implement 101 is achieved as discussed above. Thus, the ability of the surgeon to reach the cut location by positioning the implement 101 under the membrane 475 is a more practical undertaking due to the curved nature of the implement 101. The surgeon is no longer reliant on a straight, blunt device in an attempt to achieve the desired orientation to make the cut. This also means that the risk of unintentional injury to the retina or optic nerve 460 from the implement 101 may be lessened.

In the embodiment shown, the surgery is aided by a light instrument 425 through another cannula 430 providing light 440 to the eye interior 310. Notice that both illustrated cannulas 315, 430 are in offset locations through the sclera 370 to avoid more delicate cornea 490 and lens 480 features. In addition to minimizing injury and heal time, this offset point of entry also provides an added orientation benefit to the surgeon. Specifically, by entering from an offset position, the curved implement 101 is provided the opportunity to traverse a greater distance across the eye interior 310 in reaching the cut location. This means that upon reaching the cut location at an opposite side of the eye interior 310, the implement will be well positioned to make the desired cut in the membrane 475. Indeed, for circumstances where the cut location might be opposite the depicted location, the light instrument 425 and implement 101 may switch cannulas 315, 430. Of course, this is only exemplary and cannula positioning may be predetermined based on where the target cut location is within the eye 350. So long as the curved nature of the implement 101 is available appreciable benefit may be realized that is only enhanced by the commonly employed offset positioning of cannulas 315, 430.

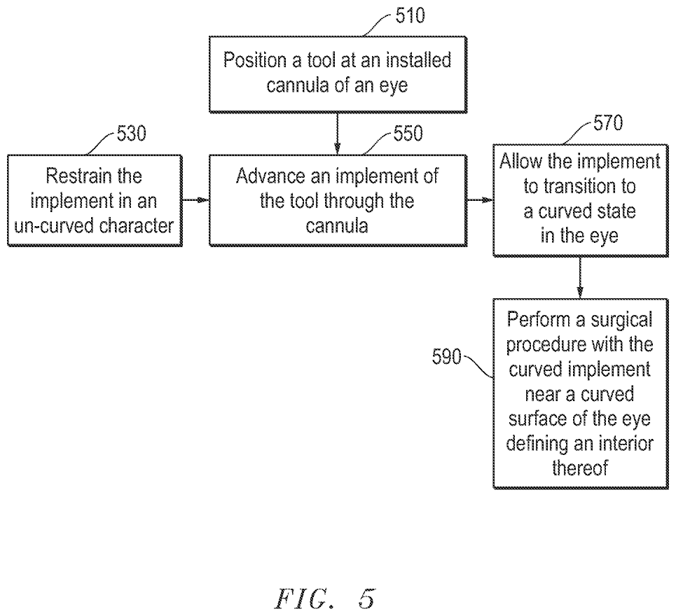

Referring now to FIG. 5, a flow-chart is shown summarizing an embodiment of utilizing a surgical tool with a curved memory shape implement to perform surgery in an eye. The implement is introduced to the eye through a cannula as indicated at 510. Whether through the aid of a sleeve or more directly by way of the cannula itself, the implement may be restrained in a more linear fashion as noted at 530 so that it may traverse the channel of the cannula (see 550). Thus, the implement may reach the interior of the eye where it is allowed to take on the curved memory shape as indicated at 570. In this way, a surgical procedure may take place that is enhanced by the use of a curved implement (see 590).

Embodiments described hereinabove include a mechanism that allows for the use of a curved instrument or implement inside a patient's eye without the need to employ larger cannulas to accommodate the curved morphology of the implement. Instead, the surgical tool is of a shape memory character that allows for straightening while accessing the interior of the eye. This means that the surgeon is presented with a manner of utilizing a curved implement at the interior of the eye instead of having to rely on a straight tool that might present an ergonomic challenge to the procedure.

The preceding description has been presented with reference to presently preferred embodiments. However, other embodiments and/or features of the embodiments disclosed but not detailed hereinabove may be employed. Furthermore, persons skilled in the art and technology to which these embodiments pertain will appreciate that still other alterations and changes in the described structures and methods of operation may be practiced without meaningfully departing from the principle and scope of these embodiments. Additionally, the foregoing description should not be read as pertaining only to the precise structures described and shown in the accompanying drawings, but rather should be read as consistent with and as support for the following claims, which are to have their fullest and fairest scope.

1. A curved tool for surgery in an eye, the tool comprising:
a handle for manipulation by a surgeon during the surgery;
an implement coupled to the handle for reaching an interior of the eye through a preplaced cannula at an outer eye surface;
a curved shape memory region of the implement corresponding to a curved shape surface defining an interior of the eye, the region straightenable for traversing the cannula and returnable to the curved shape upon the traversing; and
an edge coupled to the shaped memory region for contacting a feature of the eye at the curved shape surface, the edge having a rigid character greater than that of the shaped memory region.

2. The curved tool of claim 1 wherein the edge is a cutting feature.

3. The curved tool of claim 2 wherein the curved shape memory region and edge comprise scissors.

4. The curved tool of claim 1 further comprising a sleeve to retain the implement in advance of the traversing.

5. The curved tool of claim 1 wherein the shaped memory region is of a nickel titanium alloy.

6. The curved tool of claim 1 wherein the edge is comprised of stainless steel.

7. The curved tool of claim 1 wherein the curved shape memory region is less than about 5 mm (millimeters).

8. A curved tool for surgery in an eye, the tool comprising:
an implement for reaching an interior of the eye through a preplaced cannula at an outer eye surface;
a curved shape memory region of the implement, the region straightenable for traversing the cannula and returnable to a substantially curved shape upon the traversing; and
a flat portion of the curved shape memory region for contacting a feature of the eye at the curved shape surface, the flat portion having a rigid character greater than that of the shaped memory region.

9. The curved tool of claim 8 wherein the implement comprises scissors and the curved shape memory region is a blade thereof.

10. The curved tool of claim 8 wherein the flat portion comprises a cutting edge of the blade.

11. A method of performing surgery in an eye, the method comprising:
positioning an implement of a surgical tool at an installed cannula at the eye;
restraining a curved shape memory region of the implement in a straightened manner during the positioning;
advancing the implement through the cannula into the eye; and
returning a curved shape to the curved shape memory region with the implement in the eye.

12. The method of claim 11 further comprising performing a surgical procedure at a curved surface defining an interior of the eye with the curved shape memory region of the implement.

13. The method of claim 12 wherein the surgical procedure includes cutting with the curved shaped memory region.

14. The method of claim 13 wherein the curved shape memory region is coupled to one of an edge and a flat region having a rigid character greater than the curved shape memory region.

15. The method of claim 14 wherein the curved shape memory region is of a nitinol material and the rigid character is supplied by stainless steel.
